(19) United States
(12) Patent Application Publication
(10) Pub. No.: US 2026/0233129 A1
(43) Pub. Date: Aug. 13, 2026

(54) APPARATUS AND PROCESS FOR RECOVERY OF A VALUABLE MATERIAL

(71) Applicant: Evonik Operations GmbH, Essen (DE)

(72) Inventors: Michael Hay, Heusenstamm (DE); Henning Kaemmerer, Kleinostheim (DE)

(73) Assignee: Evonik Operations GmbH, Essen (DE)

(21) Appl. No.: 19/158,314

(22) PCT Filed: Feb. 15, 2024
(86) PCT No.: PCT/EP2024/053808

§ 371 Date: Aug. 20, 2025

(30) Foreign Application Priority Data
Feb. 22, 2023 (EP) 23157996.2

(51) Int. Cl. B01D 25/21; B01D 25/28; C07C 319/20; C07C 319/28

(52) U.S. Cl. CPC B01D 25/215; B01D 25/285; C07C 319/20; C07C 319/28

(57) ABSTRACT

Membrane filter press comprising two end elements and a plurality of filter elements, wherein a main space which is delimited from a rear back or filtrate space by a filter medium is in each case formed between two filter elements and associated frame sections, wherein at least one filter element is formed as a membrane filter element by two filter elements forming a main space and comprises a fluidizable interior which is delimited by at least one flexible membrane in the direction of the filter medium and wherein the main space has at least one feed channel for a substance mixture to be separated and the back or filtrate space comprises at least one filtrate channel for the filtrate, in particular at least one filtrate channel which leads to a collecting channel and wherein the main space comprises not only the feed channel but also at least one discharge opening, in particular a discharge opening arranged vertically above the feed channel. The invention further comprises a plant and a process for producing a solid product and/or valuable material.

The invention relates to a membrane filter press, to a plant and to a process for improved separation of a solid product or a valuable material.

A multiplicity of processes and apparatuses for separating solid products and/or valuable materials from auxiliary products or byproducts in a single- or multi-stage treatment stage downstream of the actual chemical reaction are known in the chemical industry. These generally employ filtration or separation apparatuses that operate on a continuous or batchwise basis.

Batchwise-operating vertical filter presses or membrane filter presses having filter plates hanging next to one another exhibit high dehumidification rates and customary embodiments for solid-liquid filtration are known for example from DE 20 2021 101 89 U1, DE 20 2018 104 129 U1 or DE 10 2015 007 535 A1. A membrane filter press comprises up to 200 membrane filter plates which are arranged parallel to one another between a head plate and an end plate. It is also known to combine rigid filter plates without a membrane with membrane filter plates to afford a plate package. The two lateral surfaces of the membrane filter plates are provided with flexible or movable membranes and are driven and restored by flooding of an interior space with, for example, water. This step is used for pressing and dewatering the resulting filter cake at the end of the loading phase before the membrane filter press needs to be opened and emptied. The membranes may form a single, monolithic component with the membrane filter plate or may be replaceable components. The membrane filter press, as disclosed in DE 20 2018 104 129 U1, has a central feed and each plate has a central opening through which the substance mixture to be separated (feed) can flow from one filter space to the adjacent filter space. The four collecting channels for discharging the filtrate are formed by channel segments in each plate which in the closed formation of the membrane filter press form the respective common flow channel.

DE 10 2007 027033 B4 discloses a membrane filter press which allows a more far-reaching treatment of the filter cake by allowing undesired substances to be washed out through rinsing. To this end the plates have a continuous rinsing channel on two plate edges. An introducing element leads from the supplying rinsing channel into the respective filter space and a discharging element leads to the discharging rinsing channel and out of the filter space without crossing the filter medium. The filter cake may thus be rinsed for example with water or a liquefaction medium and undesired adhering substances discharged.

In the production process for methionine which is common knowledge it is known to effect a discharging and treatment of the mother liquor from the production of methionine using a filter apparatus through which unreacted liquid substances and inhibitors are discharged. The amino acid methionine is employed in many fields, for example pharmaceutical, health and fitness products, but particularly as a feedstuff additive in many feedstuffs for various livestock. On an industrial scale, methionine is produced chemically via the Bucherer-Bergs reaction, which is a variant of the Strecker synthesis. The starting substances 3-methylmercaptopropanal (MMP, produced from 2-propenal and methylmercaptan), hydrocyanic acid (hydrogen cyanide), ammonia and carbon dioxide are reacted to afford 5-(2-methylmercaptoethyl) hydantoin (methionine hydantoin) and this is subsequently subjected to alkaline hydrolysis with alkali metal hydroxide and/or alkali metal carbonate and alkali metal hydrogencarbonate, for example potassium hydroxide and/or potassium carbonate and potassium hydrogen carbonate, to afford alkali metal methionate (for example potassium methioninate) (see formula 1).

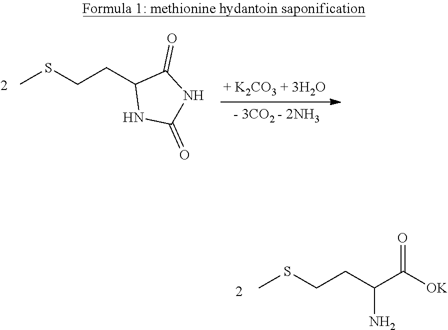

The solid product methionine is finally liberated from its alkali metal salt by acidification, for example with carbon dioxide (carbonation) (see formula 2) and is filtered off as a precipitate from the suspension containing alkali metal carbonate and alkali metal hydrogencarbonate (for example potassium carbonate and potassium hydrogencarbonate).

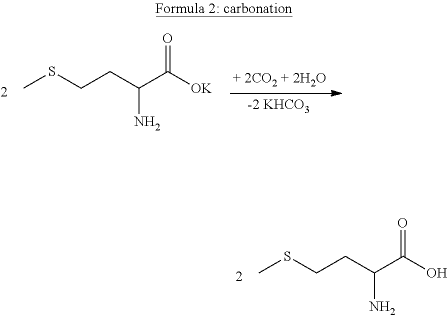

Filtration is carried out continuously, for example using a separation or filtration unit, wherein the filtrate, the mother liquor containing alkali metal hydrogencarbonate (e.g. potassium carbonate and potassium hydrogencarbonate) (1st mother liquor), is recirculated to saponify methionine hydantoin. Such a process is disclosed for example in EP 780 370 A2.

As is the case with all recirculating processes this process too requires a discharging of the filtrate to ensure that individual precursors and byproducts formed, in particular formate, do not increase beyond an acceptable level since these are inhibitors. However, the discharged filtrate still contains the product methionine, alkali metal carbonate and alkali metal hydrogencarbonate (for example potassium carbonate and potassium hydrogencarbonate) which is useful for the saponification of methionine hydantoin. To recover as much methionine and potassium hydrogencarbonate as possible from the filtrate the filtrate to be discharged may be subjected to a second carbonation.

Obtaining methionine and potassium hydrogencarbonate from the 1st mother liquor via a second carbonation is the subject of various processes as disclosed for example in DE 2 421 167 A1 in which separation after carbonation via a centrifuge is proposed. Alternatively, EP 839 804 A2 proposes using a water-soluble liquefaction medium to precipitate the methionine which entails additional effort and possible impurities.

Even if the separation apparatuses are known and tested the separation step often requires extensive cleaning of the separation apparatus with a high degree of manual effort, as is especially true for separation apparatuses such as filter or membrane filter presses which are capable of achieving particularly high drying values.

It is accordingly an object of the present invention to provide an improved separation apparatus which makes it possible to achieve higher performances for a plant or a process for a solid product and/or a solid valuable material.

This object is achieved by a plant for producing a solid product and/or valuable material having the features of claim 1 and a process for producing a solid product and/or valuable material having the features of claim 10.

The plant is configured for producing a solid or solidifiable product and/or valuable material, in particular for producing the at least one product and/or at least one valuable material in a liquid substance mixture, comprising a process unit and at least one connected separating unit, wherein the process unit comprises the following:
at least one reactor for precipitating or solidifying the valuable material,
at least one feed conduit to the processor unit and/or the reactor for introducing at least one reactant and, if required, further (auxiliary) substances,
a connecting conduit for discharging a suspension comprising at least one precipitated, solid valuable material as a liquid substance mixture to the separating unit, wherein the separating unit is configured for separating the solid valuable material from the suspension. The separating unit comprises at least one membrane filter press, wherein the membrane filter press is connected via
   a conduit to a receiving and conditioning unit and via
   a return conduit to the process unit.

In the present document “a solid product and/or a solid valuable material” is referred to collectively as “valuable material”, wherein valuable materials are to be understood as meaning substances or mixtures thereof which in the case of recycling into the process upstream of the plant for separating the valuable material as solid and/or the liquefaction according to the invention have a positive effect on the process in the absence of specific indications or indications to the contrary. In particular, a valuable material is not an inhibitor or contaminant and/or is not disadvantageous to the respective process and/or to product quality. In the present case valuable materials may accordingly be selected for example from reactants, byproducts, auxiliaries and/or end products of the respective process for the end product. The product and/or the valuable material are especially chemical substances or substance mixtures. In the present case “byproduct” is also used synonymously for all substances in a substance mixture which do not constitute solid “valuable material” in the absence of specific indications.

In a first embodiment the receiving and conditioning unit is a heat exchanger or a heating apparatus, by means of which the liquefaction medium is brought to a temperature which is at least equal to the melting or dissolution temperature of the solid valuable material, ideally at least 5% above the liquefaction or dissolution temperature of the solid valuable material. In the case of production of methionine and liquefaction thereof it is advantageous to establish a temperature of not less than 60° C., ideally not less than 70° C.

The receiving and conditioning unit may also be an adjacent process unit or plant unit from which a suitable liquefaction medium is derivable.

The term “reactant” is to be understood as meaning all starting materials and auxiliaries and optionally flowable or liquid catalysts which are in simplified terms collectively referred to as reactant. The states of matter generally refer to standard conditions of 1 bar and 20° C. or process-typical conditions such as are known to those skilled in the art outside the invention in the absence of indications to the contrary.

Furthermore, the numerical indication of “a” unit, such as “a reactor” shall be understood to also encompass two or more units and not necessarily be limited only to “a single” unit.

Finally, the term “connected” is to be understood as meaning not only a direct connection but also a conduit or conduit connection in which units of secondary importance for the present invention are arranged, such as for example valve manifolds, a stack tank or an indirect heat exchanger.

The membrane filter press comprised by the plant, comprises the following:

Two end frames, a plurality of filter elements arranged between the end frames, at least one feed channel for a suspension and at least one collecting channel for discharging the filtrate, wherein a filter chamber which is divided into a main space and a rear filtrate space by an insertable filter medium is in each case formed between two filter elements and the associated frame sections. At least one filter element is formed as a membrane filter element by two filter elements forming a main space and has a fluidizable interior. The fluidizable interior of a membrane filter element may also be formed from a plurality of sub-spaces which are interconnected via channels or conduits. The at least one interior is delimited by at least one flexible membrane in the direction of the main space/of an inserted filter medium. The flexible membrane ideally forms at least one wall or wall section of the interior and may be moved, stretched, or expanded in the direction of the main space by increasing the pressure in the interior. The main space has at least one feed channel for a substance mixture to be separated or is connected therewith. The back or filtrate space has at least one filtrate channel for discharging the filtrate or is connected to such a filtrate channel, wherein advantageously the at least one filtrate channel leads to the collecting channel, by means of which the filtrate is dischargeable from the membrane filter press. The collecting channel is advantageously formed from a multiplicity of channel segments, each of which are arranged in a filter element and together form the overall collecting channel.

The main chamber comprises not only the feed opening but also at least one discharge opening, particularly a discharge opening arranged vertically above the feed channel.

In other words it is possible to establish direct traversal of the main space with a liquefaction medium from the feed opening for the substance mixture to be separated to the additional discharge opening without passage of the liquefaction medium through the filter medium, in particular not to a filtrate channel and/or collecting channel for the filtrate. This is also to be distinguished from known filter presses or membrane filter presses where the main space with the formed filter cake may be rinsed by injecting via upper introducing rinsing channels for example a fluid that is discharged again via lower discharging rinsing channels, the feed opening, or feed channel not being integrated here.

A filter element may In principle have any polygonal, round, or elliptical shape. A filter element advantageously has a square, rectangular or up to octagonal basic shape. Said element has lower frame sections, upper frame sections and lateral frame sections. In the case of square filter elements these are a lower frame section, an upper frame section and two lateral frame sections with a frame or edge region. Although the membrane filter press and the formed spaces and their flow channels are described with reference to the position of the filter medium and the membrane filter press is only usefully operable with filter media the filter medium is a wear part and is generally not part of the membrane filter press.

The terms “fram” or “edge regio” are not to be understood as geometrically restricted and particularly refers to the region of the filter element that is in contact with an adjacent filter element and/or to which the filter medium is secured.

The term filter element is moreover to be understood as also encompassing a group of especially frame- or plate-type elements which together form a filter element, in particular a group of elements which are stacked against one another parallel to the planes spanned by the filter medium and together form one filter element and also comprise corresponding flow channels, in particular internal flow channels. Thus, for example two circumferential elements or frames may each bear a filter medium and a central element forms a dividing and supporting wall so that the three elements together form one filter element.

In analogous fashion a circumferential element or frame may in each case bear a filter medium and the central element has at least one expandable membrane and itself comprises no filter media, the group of the elements together forming a complete membrane module.

Further aspects of the basic construction of filter presses or membrane filter presses and further details thereof are altogether known, such as the outer supporting structure, sealing means, inner geometries, drive means for opening, closing and pressing the filter elements, different sequences of membrane filter frames and filter frames, discharging apparatuses, connection elements for media to the head plate, etc., so that these and further aspects may be augmented and/or combined by a person skilled in the art as desired. This also applies to the process engineering requirements for operating a filter or membrane filter press. In particular one of the two end elements, the head plate/element, comprises at least one connection element, for example a flange or a quick-action coupling for each (inner) channel or conduit.

In known fashion the head element is in particular the idle side and the opposite end element the movable side which is moved with a motor to seal and to press the membrane filter press.

In an advantageous embodiment it may be provided that the at least one feed conduit is arranged at a vertically lower frame section. This has the advantage that upon introducing a feed the trapped gas is passed upwards, for example to the discharge opening or a filtrate channel and may be released.

Furthermore, during the step of liquefaction, the filter cake is arranged vertically downward of the introduced liquefaction medium so that the discharge opening is largely kept free and the most intensive possible mixing of the solid (filter cake/filter cake pieces) with the inflowing liquefaction medium is effected.

In a further advantageous embodiment, it may thus analogously be ensured that the at least one discharge opening arranged vertically above the feed channel is arranged at the vertically upper frame section.

Since the liquefaction medium must bridge the main space largely filled with filter cake, i.e. experiences a high flow resistance at least temporarily, it may be provided in a further advantageous embodiment that the number of the lower feed channels or supply channels is at least one greater than the number of outflows/discharge channels.

It has proven particularly advantageous when two lower feed openings and/or feed channels and one upper discharge opening/one upper discharge channel are provided in a filter element.

It is thus provided in a further advantageous embodiment that the ratio of the sum of the flow cross sections of the feed channels to the sum of the flow cross sections of the discharge channels is in the range from 1:3 to 2:1, ideally in the range from 1:2 to 1:1. This ensures that fresh liquefaction medium can flow in at any time without excessive flow resistance and further that smaller fragments of the filter cake are discharged as solid and optionally completely dissolved during subsequent transport.

It is advantageous when the separating unit comprises two, three or four filter apparatuses and/or two, three or four subunits comprising a precipitation reactor and a subsequent filter apparatus which are connected in series for optimal discharging of the valuable material. Depending on the valuable material to be separated it is possible for filter apparatuses to be serially connected with increasing fineness, i.e. with decreasing average mesh size or average pore size, and/or for a plurality of precipitation steps with respective subsequent solid-liquid separation to be carried out, in particular using filter apparatuses. It is apparent to a person skilled in the art that depending on the volume flow to be treated the separating unit may comprise a plurality of parallel flow paths for partial volume streams to separate the valuable material from the respective partial volume stream, wherein each flow path for a partial volume stream may analogously comprise subunits, precipitation reactors and/or filter apparatuses.

In an improvement of the plant, it may be advantageous for the membrane filter press to be configured according to one of the aforementioned embodiments and/or variants.

It may especially be advantageous when the last subunit and/or the last filter apparatus is the membrane filter press according to the invention or an analogous filter press which may be emptied and/or regenerated using a liquefaction medium.

It may be advantageous when at least two of the flow paths of the separating unit, each of which conduct a partial volume stream, are connected to a common membrane filter press according to the invention which may be emptied and/or regenerated using a liquefaction medium. It may altogether be particularly advantageous when, of 1 to n filtration or separation apparatuses for a valuable material along the main processing or main flow direction, the n-th filter apparatus, i.e. the final filter apparatus, is the membrane filter press according to the invention. All aspects and advantages that have been recited for the membrane filter press shall in identical fashion or in analogous application also apply to the plant and vice versa unless these aspects and advantages are ruled out for physical or chemical reasons.

In a further improvement of the plant it may be advantageous for the membrane filter press to comprise a plurality of filter elements, wherein a main space is in each case formed between two adjacent filter elements, wherein at least two main spaces are connected to at least one discharge channel of the membrane filter press via the respective discharge opening and wherein the at least one discharge channel is connected to the process unit, in particular a reactor of the process unit. The discharge channel is advantageously connected to a container or stack tank arranged upstream of the process unit.

The collecting conduit is in particular an inner collecting conduit, wherein “inner” collecting conduit is to be understood as meaning that said conduit is formed from channel segments of the individual filter elements and advantageously each channel segment is an integral constituent of the respective filter element.

The connection of the collecting conduit with the process unit or a reactor of the process unit may also be effected via workup stages, intermediate treatments, etc.

In a further improvement of the plant, it may be advantageous when the feed channel/the feed openings to the respective main spaces of the membrane filter press are connected as follows, namely
I. to a filtrate-conducting conduit (outflow) of an upstream separation apparatus,
II. to a flow path (outflow) that is connected to the outflow of the process unit, in particular a direct flow path, and/or
III. to a receiving and conditioning unit for providing a liquefaction medium, in particular a separate receiving and conditioning unit. Here, “separate” is to be understood as meaning that said unit is not integrated into the main process and is connected to the main process only for the step of liquefaction/regeneration of the separation apparatus.

In alternative I it may especially be the case that the membrane filter press is the last filter apparatus of the plant at position n and the filtrate which is used as liquefaction medium and is conditioned if required originates upstream from the filter apparatus of position n−1. In this case the number of filter apparatuses n may in particular be 2, 3, 4 or 5. It may especially be advantageous when at least one or more of the filter apparatuses at positions 1 to n−1 are continuously operating filter or separation apparatuses.

In other words, the liquefaction medium is in particular a suspension, a substance mixture or a filtrate from one of the three following flow paths, wherein
in case I. the filtrate of an upstream separation apparatus is at least partially utilized, optionally after an additional chemical and/or physical conditioning
in case II. at least one separation apparatus of the separating unit from the flow path of the suspension or of the substance mixture to the liquefaction of the valuable material is at least partially or completely bypassed and
in case III. The liquefaction medium is withdrawn from a separate receiving and conditioning unit for liquefaction.

A critical aspect of variant I. could be that a more marked concentration of inhibitors or contaminants that could be disadvantageous during recycling into the process unit may already have taken place, wherein this depends on the respective process.

Variant III. Allows optimal conditioning and choice of the liquefaction medium but has the disadvantage that it requires the greatest construction effort. There may therefore be a particular advantage in providing variant I. for liquefaction because this allows direct utilization as liquefaction medium of the filtrate that is present in the process and has already been depleted in terms of at least one valuable material, i.e. also in terms of total volume.

In variants I. or II. a conditioning may likewise be effected on the flow path to the separation apparatus to be regenerated, for example a temperature adjustment for the purpose of optimal liquefaction.

In an advantageous configuration of the plant, said plant is used for producing methionine. Precipitation of the methionine from the substance mixture is effected by carbonation. The plant advantageously comprises at least two separation apparatuses, of which the first separation apparatus is advantageously a continuous separation apparatus, such as a centrifuge, a rotary vacuum filter apparatus or a vacuum belt filter apparatus. It is advantageous when for precipitation of the valuable material each separation apparatus has a carbonation reactor arranged upstream of it, in particular a carbonation reactor to which a gas conduit into the reactor interior, for example for CO₂ introduction, is connected.

It Is advantageous when the last separation apparatus is a membrane filter pressllch is configured according to one of the aforementioned embodiments or variants.

The invention further relates to a process for producing a solid product and/or a solid valuable material using a plant, in particular for producing the at least one product and/or at least one valuable material in a liquid substance mixture, comprising the following steps:
a. provision of a process unit and a separating unit,
b. introduction of at least one reactant and, if required, further (auxiliary) substances into the process unit, production and precipitation of at least one solid valuable material in a suspension, in particular precipitation downstream of the production/of the unit and/or of the site of production (synthesis),
c. separation of the at least one solid valuable material and discharging of the at least one byproduct using at least one separation apparatus in the separating unit,
d.

This comprises undertaking the following steps:
e. collection of at least one sub-amount of the separable valuable material as filter cake in the at least one separation apparatus. Collection is here in particular to be understood as meaning the temporary enrichment, accumulation, or filling of the valuable material in the inside of the separation apparatus through inflow via at least one feed channel. This causes the pressure inside the separation apparatus and in particular the main spaces to increase while displaced gas and/or filtrate is simultaneously expelled. The filtrate and at least temporarily also the gas passes through the respective filter media and exits the separation apparatus via the filtrate channels and advantageously via at least one collecting conduit. The gas may also be discharged via other (gas) channels which are temporarily or permanently suitable therefor such as for example a discharging channel for a liquefaction medium specified below which is empty in this process step.
f. dehumidification of the at least one sub-amount of the valuable material in the at least one separation apparatus, wherein advantageously the dehumidifying of the valuable material is carried out at least up to a residual moisture content of less than 60%, ideally to a residual moisture content of not less than 35% to not more than 10%. The dehumidification may be carried out in one or more stages, in particular also pneumatically through introduction of a gas or gas mixture and/or mechanically via a fluidically generated pressure. The pressure in the mechanical dehumidification is advantageously generated with an incompressible medium, in particular using a liquid which presses a pressing membrane in the direction of the filter cake (valuable material).
g. liquefaction of the at least one sub-amount of the valuable material collected in the at least one separation apparatus and solidified as a filter cake inside the separation apparatus with a liquefaction medium.
   The “term “inside the separation app” ratus” is to be understood as meaning that the valuable material to be liquefied is not discharged and the separation apparatus is thus also not opened after the collection and the single- or multi-stage dehumidification. The liquefaction medium is advantageously introduced into the main spaces of the filter elements of the membrane filter press via one or more feed channels and feed openings after complete filling (collection).
h. recycling of the liquefied valuable material into the process unit and/or discharging into a stack tank.

An alternative variant of this step and thus of the process may provide that the valuable material dissolved in the liquefaction medium is at least temporarily stored in a tank, in particular intermediately stored and subjected to further conditioning if required before introduction to a location upstream of the separation unit having regard to the main production process.

In an alternative embodiment of the process, step f. of liquefaction may be preceded by an emptying step where the separation apparatus is opened and the filter cakes or the portion of the filter cakes are discharged by gravity, i.e. fall out, in particular to the extent that this is possible without manual and/or mechanical intervention. In other words, the emptying step of these process variants comprises undertaking an incomplete emptying and greater manual cleaning effort is eschewed.

The separation apparatus is then closed again, sealed by pressing and the liquefaction of the remaining adhering filter cake or fragments thereof is undertaken. It may be advantageous when at least the sealing surfaces are very largely freed of deposits, for example using a brush or compressed air, before closing of the separation apparatus and liquefaction.

In a further improved process variant, it may be provided that the dehumidification of the valuable material is carried out in at least two serially connected separation apparatuses in at least two sub-steps, wherein
in the first sub-step a first sub-amount of the valuable material is separated and dehumidified, wherein the filtrate separated in the first separation apparatus is sent on into the second separation apparatus and wherein
in the second sub-step the valuable material present in the filtrate is separated and dehumidified.

It may additionally be advantageous when the first separation apparatus is a continuously operating separation apparatus, in particular a vacuum filter, such as for example a rotary drum filter or a belt filter, or a continuously operating centrifuge or another centrifugal separator, and wherein the second separation apparatus is a chamber filter press, in particular a chamber filter press or a membrane chamber filter press.

In a further improved process variant it may be provided that the dehumidification in the first sub-step may be carried out to a residual moisture content in the separated valuable material of 60% to 30% and the dehumidification in the second sub-step is carried out to a residual moisture content in the separated valuable material in the range from 45% to 10%. The particular separation performance is very dependent on the respective substances to be separated and the separation apparatuses. Ideally the first sub-step comprises a dehumidification to a residual moisture content in the separated valuable material of 60% to 40% and the dehumidification in the second sub-step is carried out to a residual moisture content in the separated valuable material in the range from 35% to not more than 15%, ideally in the range from 30% to 10%.

It may altogether be advantageous when the filter medium is spared the burden of passage of a liquefaction medium. In an improved process variant, it is therefore provided that during feeding of a liquefaction medium into the main space and discharging via one or more discharging channels a pressure elevation is effected in the filtrate space or rear space. This pressure in the filtrate space or rear space should be at least equal to or slightly above the pressure in the main space and may be effected by introduction of a fluid, in particular of a gas. To prevent precipitations the fluid is advantageously heated, in particular to the temperature prevailing in the main space/to the temperature of the liquefaction medium.

In a further improved process variant it may be provided that the liquefaction medium for liquefaction of the valuable material collected inside the at least one separation apparatus is an acid and/or an alkali, in particular an acid or alkali which is not a substance mixture, not a filtrate or not a suspension, i.e. which was not withdrawn from the plant or the process at an upstream location.

It may be advantageous when the liquefaction comprises a conditioning of the liquefaction medium, in particular a heating and/or adjusting of the pH value. The adjusting of the pH is carried out in known fashion by addition of an acid or alkali. It is particularly advantageous when the liquefaction medium is derived as filtrate from an upstream process stage to the separation unit, wherein it may be advantageous when the filtrate is conditioned for the purpose of dissolving the valuable material.

In analogous fashion it may be advantageous when before introduction into the second separation apparatus/the second separation step the filtrate from the first separation apparatus is supplied to a conditioning unit for performance of a conditioning step where for more far-reaching precipitation of the valuable material and the filtrate:
a post-reaction with/by addition of a reactant and/or
alteration of the process parameters such as for example temperature, pressure is effected.

In a further improved process variant it may be provided that during the liquefaction the (separated) valuable material collected inside the at least one separation apparatus is subjected to a flow of the liquefaction medium from below in the gravitational direction and is discharged from the respective main space and the respective filter element at the top and/or above the feed channel in the gravitational direction.

The introduction of the liquefaction medium is ideally effected via the at least one feed channel for the feed. A conduit connection, port, connecting element, valve, etc. may be provided for this purpose.

It is particularly advantageous that parts or fragments of the optionally dissolved but not yet liquefied valuable material fall towards the inflowing fresh liquefaction medium while the discharge opening is kept free. By contrast, small fragments of the still solid valuable material may be entrained which may be intentional.

In a development of the process a sieve or a grid structure may be provided in or upstream of the discharge opening to specify the maximum size of fragments of the filter cake/the valuable material that may be washed out.

It is altogether advantageous when the progress of the liquefaction is monitored using sensors. This may be effected for example via measurement of the flow rate, the pressures, the pressure difference, the vibrations, the conductivity, the capacitive loading, or the progress thereof over time. Especially in batch operation it is advantageous to be able to determine the end or the extent of liquefaction, and thus the output of the valuable material, to allow termination of the cycle. Every main chamber or every discharge opening advantageously comprises such a sensor. This allows the separation apparatus to be opened at a defined point and an individual, faulty filter element to be replaced as required. This is especially advantageous because, in contrast to a typical operating mode of a membrane filter press, constant optical inspection by an operator manually emptying the filter press is not carried out.

Process according to any one of the preceding process claims, characterized in that the process is used for producing the solid valuable materials methionine and/or methionyl-methionine (dipeptide of methionine; met-met).

The liquefaction medium is advantageously a substance mixture from an upstream unit, in particular the substance mixture is the outflowing suspension from the first or the second carbonation or the filtrate from an upstream separation apparatus, such as a continuous filter apparatus. A preceding conditioning, which may in particular consist of a temperature elevation, may be provided. The temperature of the substance mixture is advantageously increased to above 65° C., ideally above 75° C. The temperature of the liquefaction medium is not more than 115° C., ideally 110° C.

It may altogether be advantageous when a membrane filter press and/or a plant configured according to at least one of the herein recited exemplary embodiments or variants is provided for the process.

All aspects and advantages that have been recited above for the membrane filter press or the plant shall in identical fashion or in analogous application also apply to the process and vice versa unless these aspects and advantages are ruled out for physical or chemical reasons or are inapplicable.

The invention is more particularly described hereinbelow with reference to exemplary embodiments and figures. In the figures:

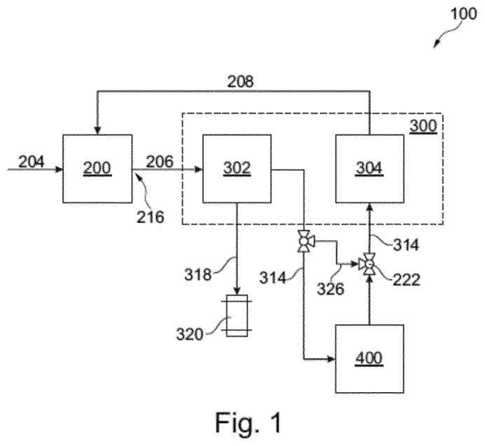

FIG. 1 shows a first exemplary embodiment as a schematic diagram of a plant and a process,

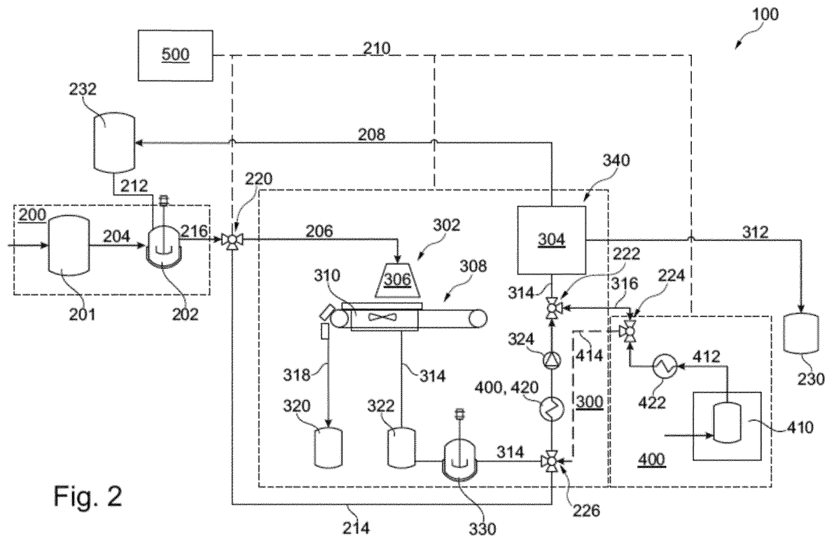

FIG. 2 shows a further exemplary embodiment as a schematic diagram of a plant and a process,

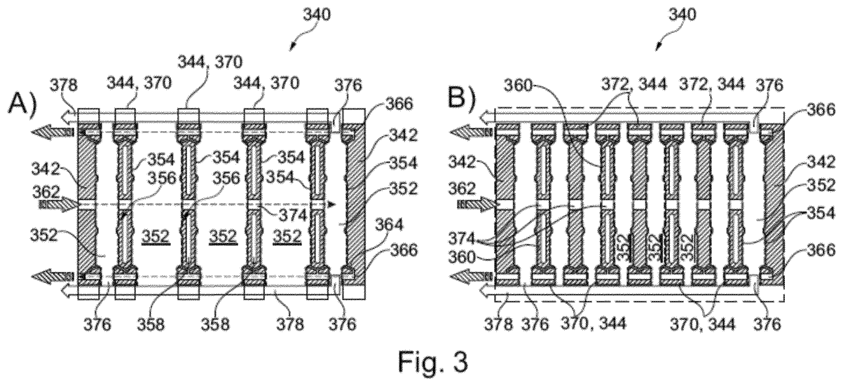

FIG. 3 shows two exemplary embodiments for membrane filter presses in two partial representations A) and B),

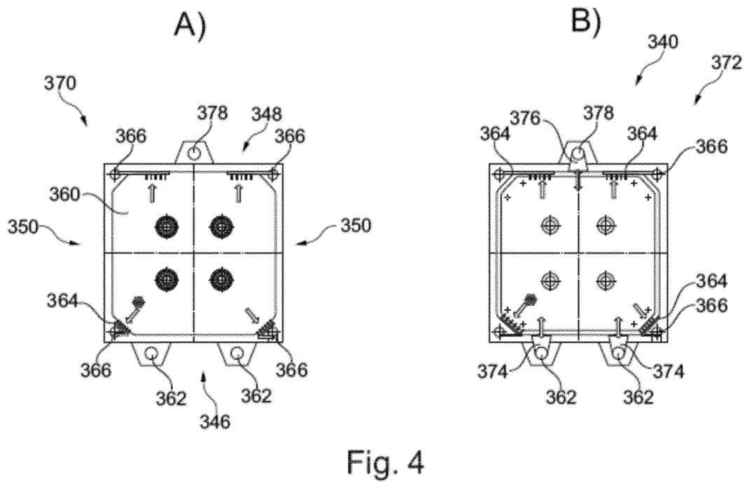

FIG. 4 shows a further exemplary embodiment of a membrane filter press in two partial representations A) and B),

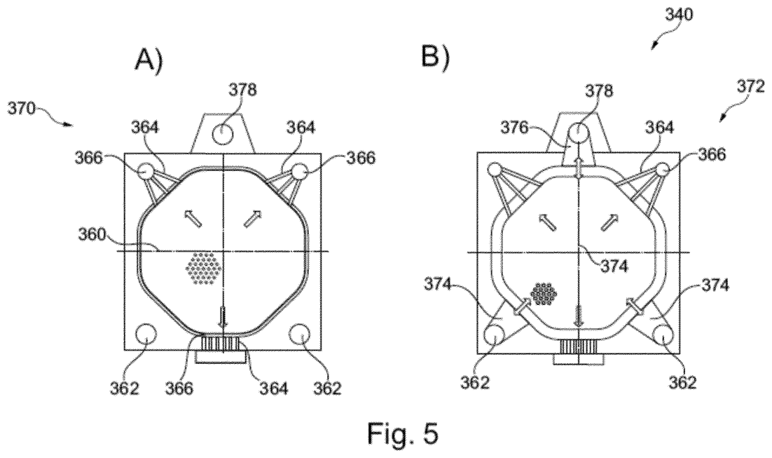

FIG. 5 shows a further exemplary embodiment of a membrane filter press in two partial representations A) and B) and

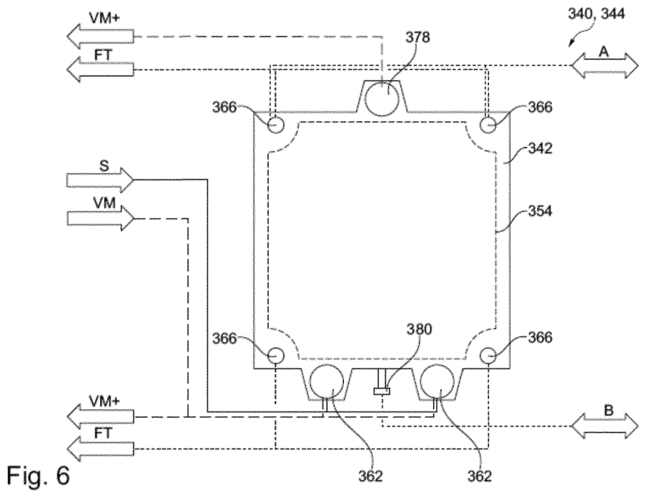

FIG. 6 shows a schematic representation of the different feeds on an end or head element.

FIG. 1 shows a plant for producing at least one solid or solidifiable valuable material. The plant 100 comprises a process unit 200 having a feed conduit 204 for reactants not further specified or distinguished and an outflow 216. For workup of the liquid substance mixture the outflow 216 is connected to a separating unit 300 via the connecting conduit 206. The separating unit 300 comprises a first separation apparatus 302 and a second separation apparatus 304. The first separation apparatus 302 comprises a discharge conduit 318 for a valuable material, in particular a solid valuable material, and a connecting conduit 314 to the second separation apparatus 304, into which the filtrate from the first separation apparatus 302 is conveyed. A receiving and conditioning unit 400 is provided in the connecting conduit 314 and upstream of the second connecting conduit 304. The depleted substance mixture, for example a filtrate from the first separation apparatus 302, may be altered in terms of its chemical and/or physical properties using the receiving and conditioning unit 400 in order to achieve more far-reaching substance separation. A further solids precipitation is especially carried out to this end. The second separation apparatus 304 is connected to the process unit 200 via a (recycling) conduit 208. The conduit 314 connecting the two separation apparatuses 302, 304 comprises a bypass conduit 326 by means of which the receiving and conditioning unit 400 can bypassed.

A more far-reaching precipitation of a solid may be undertaken by for example reducing the temperature using indirect heat exchangers and, depending on the substance, precipitating a crystalline solid. The solid is especially identical or largely identical to the solid previously separated in the first separation apparatus 302. Alternatively, the separation stage 400 comprises a feed conduit for a reactant and a mixing unit and/or a reactor to achieve a chemical reaction and precipitation of at least one of the substances conducted in the conduit 314. The solid substance to be precipitated in the flow path from the first separation apparatus 302 to the second separation apparatus is especially identical to the valuable material of the processor unit 200/the separated solid substance or valuable material from the first separation apparatus 302.

In the example shown according to FIG. 2 in the second separation apparatus 304 the suspension is further depleted in respect of the valuable material and the (second) filtrate is discharged via the conduit 312 and stacked in the container 230. The second separation apparatus 304 is a membrane filter press in which the solid is collected and dehumidified in batch operation.

After the separation of the solid valuable material by the first separation apparatus 302 and discharging of the at least one filtrate as a substance mixture to the second separation apparatus 304 a first process step comprises collecting the precipitated valuable material in the second separation apparatus 304 as a filter cake, wherein a further conditioning of the filtrate and precipitation of the solid valuable material has optionally been carried out beforehand. A further process step comprises performing a single- or multi-stage dehumidification of the separated valuable material in the second separation apparatus 304 where the enclosed filter cake is for a duration subjected to gas, in particular air or an inert gas, under elevated pressure in the filter space and/or in the main space and/or mechanically pressed via a hydraulically activatable membrane 360 (FIG. 3).

This frees the valuable material from the adhering liquid phase, and thus also very largely discharges harmful byproducts and inhibitors, wherein the valuable material has a residual moisture content of not more than 30% to 15%.

Filtration by means of membrane filter presses is highly effective and liquid contaminants or inhibitors may be efficiently separated from the filter cake in this way. However, the emptying of a membrane filter press is generally very time intensive and especially requires manual operations. Furthermore, depending on the type of filter cake and/or the substance mixture present it may be necessary to open the membrane filter press while taking particular protective precautions because the substance mixture or the solid filter cake comprises hazardous substances.

It is therefore provided in accordance with the invention in a subsequent process step that the membrane filter press is not opened for emptying and regeneration but rather a liquefaction of the filter cake/the solid valuable material is effected by passing a liquefaction medium through the main chambers of the membrane filter press. The liquefaction medium is advantageously passed through the main chambers of the filter elements of the membrane filter press in such a way that said medium does not or substantially does not flow through the filter media.

The liquefaction medium enriched with the liquefied valuable material (filter cake) is recycled to the process unit 200 via the conduit 208.

In the example shown in FIG. 1 the liquefaction medium is the filtrate from the first separation apparatus 302 which is conducted in the conduit 314 and has previously been conditioned in the receiving and conditioning unit 400 especially by temperature elevation such that the collected filter cake is liquefied. After the liquefaction inside the second separation apparatus 304 the filtrate enriched with valuable material/the enriched liquefaction medium is recycled into the process unit 200 via the conduit 208.

FIG. 2 shows a plant comparable to FIG. 1 and the accompanying process. The process unit 200 comprises a first reactor 201 and a second reactor 202, wherein the reactants are introduced into the first reactor 201 and at least one reaction or synthesis step is carried out. The second reactor 202 is a precipitation reactor in which for example the crystallization of the solid valuable material is initiated. To perform the precipitation or crystallization further reactants or auxiliaries may be introduced into the second reactor 202 but this is not shown in the present case. The plant 100 comprises a central control unit 500 to which the different units and apparatuses are connected via control and data lines 210.

The outflow 216 of the process unit 200 is connected via the connection conduit 206 to the first separation apparatus 302 which is in the form of a continuously operating vacuum filtration unit and comprises a vacuum apparatus 310. The suspension conducted in the connecting conduit 206 is applied via an application unit 306 to a continuously recirculating belt filter 308 which interacts with a vacuum apparatus 310 in known fashion. The separated solid valuable material having a first residual moisture content is passed over the conveying section 318 and collected in the container 320. The filtrate from the vacuum apparatus 310 is temporarily supplied via the conduit 314 to the container 322 in order subsequently to be supplied to the second, discontinuously (batchwise) operating separation apparatus 304. Provided downstream of the vacuum apparatus 310 is a reactor 330 which is supplied in a manner not further specified with reactants to achieve more far-reaching precipitation of the same solid valuable material or a different solid valuable material from the outflowing substance mixture (filtrate) of the first separation apparatus 302. The reactor 330 downstream of the first separation apparatus 302 generally precipitates the same valuable material from the filtrate in augmentative fashion. As described hereinabove in the first phase of the process the second separation apparatus 304 which is in the form of a membrane filter press 340 is supplied with the suspension via the conduit 314 and collection of the solid valuable material as a filter cake followed by dehumidification of the filter cake, as described in particular in connection with FIGS. 3 to 6, is effected.

With a view to the provision of a liquefaction medium for liquefying the filter cake in the closed membrane filter press 340, FIG. 2 shows altogether three alternative embodiments of which generally only one is permanently provided:
I. As a first alternative the liquefaction medium may be produced via the conduit 314 as a conditioned filtrate from the filtrate of the first separation apparatus 302, wherein the conditioning unit 400 consists substantially of the heat exchanger 420 arranged upstream of the second separation apparatus 304. In one variant the reactor 330 provided for the precipitation reaction is dimensioned and connected such that for the duration of the regeneration of the second separation apparatus 304 said reactor also functions purely as a buffering container and/or as part of the conditioning unit 400 for providing the required liquefaction medium for the valuable material liquefaction.
   In one variant of this alternative the reactor 330 is bypassed for the step of the liquefaction of the filter cake in the second separation apparatus 304.
II. The second alternative comprises directly supplying a sub-amount of the substance mixture or a suspension from the process unit 200 to the second separation apparatus 304 in bypass to the first separation apparatus 302. To this end a flow path which leads into the conduit 314 via the outflow 216, a first valve unit 220, a conduit 214 functioning as a bypass to the first separation apparatus 302 and a second valve unit 226 upstream of the heat exchanger 420. In this alternative the heat exchanger 420 substantially forms the conditioning unit 400.
   Advantageously a second sub-amount of the substance mixture or the suspension from the process unit 200 is simultaneously supplied to the first separation apparatus 302 and the filtrate stacked in a suitable container or a reactor until regeneration (emptying) of the downstream separation apparatus 304 via the bypass conduit 214. In a particularly advantageous process mode and plant configuration the main production process of the valuable material need not be entirely interrupted.
III. The third alternative comprises a completely separate receiving and conditioning unit 400 such as is shown at the bottom right-hand side of FIG. 2 in schematic form and framed by a dashed line. This receiving and conditioning unit 400 provides the liquefaction medium required for the liquefaction of the solid valuable material from the second separation apparatus 304 for example in the form of pure solvent, such as for example an alkali or an acid, and may effect conditioning or metered discharging thereof as required. A thus-provided solvent is, as shown, passed via a further valve unit 222 upstream of the second separation apparatus 304 into the supplying conduit 314, wherein this may also be effected at a different suitable site upstream of the second separation apparatus 304 and/or for direct introduction into the separation apparatus 304.

The receiving and conditioning unit 400 that is integrated in alternative III. and provides a further liquefaction medium is shown in highly simplified and schematic form. It comprises a receiving tank 410 with a feed conduit symbolized with an arrow, a conduit 412, a heat exchanger 422 and a valve unit 224 and optionally conveying means and further units that are not shown. The valve unit 224 is connected via the conduit 316 with the valve unit 222 arranged upstream of the second separation apparatus 304, presently the membrane filter press 340, wherein the valve unit 222 is integrated into the supplying conduit 314 to the second separation apparatus 304. The connecting conduit shown with a dashed line indicates an alternative or an additional connecting conduit 414 to the further valve unit 226 in the conduit 314. Via this connecting conduit 414 a liquefaction medium from the receiving tank 410 may be introduced with the substance mixture from the conduit 214 or the conduit 314 upstream of the heat exchanger 420 on the suction side of the pump 324 and mixed to form the final liquefaction medium.

The liquefaction medium enriched with the completely or largely liquefied valuable material (filter cake) is passed via the conduit 208 into a container 232 which is used for stacking and controlled release into the process unit 200/the second reactor 202 via the conduit 212. In an alternative process (not shown) the enriched liquefaction medium is at least partially introduced into the first reactor 201.

The container 322 into which the filtrate from the vacuum apparatus 310 is passed via the conduit 314 and at least temporarily stacked allows uninterrupted or largely uninterrupted operation of the process unit 200 and the first separation apparatus 302.

In a further exemplary embodiment (not shown) the separation apparatus 300 comprises downstream of the first filter apparatus 302 and upstream of the terminal (last) membrane filter press 340 in the flow path 314 of the filtrate one or two (further) subunits comprising at least one precipitation reactor, for example analogously to the reactor 330 and a filter apparatus. The filter apparatus of the (further) subunit is for example a continuously operating filter apparatus, such as a belt or drum vacuum filter or may be a centrifugal separator.

FIG. 3 shows as a vertical sectional representation in two partial representations A) and B) the basic construction of a membrane filter press 340 in two embodiments in which the second separation apparatus 304 is configurable according to FIG. 1 or 2. The representations are greatly simplified, so that, for example, media connections of the end or head element, load-bearing structures, drive means, fluid connections or control components are not shown since these are in principle known to those skilled in the art. In both partial representations the membrane filter press 340 is shown in an open position in which the membrane filter frames 370 are spaced apart from one another. Partial representation A) shows a membrane filter press 340 which has two edge or end elements 342, between which only membrane filter frames 370 are arranged as filter elements 344.

Both embodiments comprise a central feed channel 364 for the substance mixture to be separated (feed) and the collecting channels 366 for filtrate discharging are arranged at the top and bottom in the corners of the filter elements 344. The membrane filter press 340 and also the individual filter elements 344 may altogether be oriented and operated in any desired inclination or orientation and so indications such as “top”, “above”, “bottom” or “below” are used for simpler description and relate to a typical, advantageous orientation and setup without any intention of a general limitation. In the present case membrane filter frames 370 and (rigid) filter frames (without a membrane) 372 are shown collectively as filter elements 344. The indications concerning filter frames 372 apply analogously to the edge or end elements 342 and their interaction with an adjacent filter element 344.

In contrast to partial representation A) the membrane filter press 340 according to partial representation B) is formed from filter elements 344 which in addition to the two edge or end elements 342 alternatingly comprises membrane filter frames 370 and filter frames 372. All filter elements 344, 370, 372 are in a vertically hanging orientation.

The filter elements 344, 370, 372 have a filter medium 354 on both sides which is configured as a filter fleece or filter mat. In the embodiment shown the filter medium 354 is formed from two parallel filter mats connected via a tubular section in a manner not further specified. In the installed state this tubular section bridges the respective central feed opening 374 of the filter element 344. Two filter elements 344/a pair of the end elements 342 and the adjacent filter element 344 form a common main space 352 in which via the uninterrupted feed channel 362 and the feed openings 374 in the respective filter elements 344 the substance mixture to be separated (feed) is introduced and in which the filter cake is formed in the course of the collection. In the left-hand end element 342, also referred to as the head element, the feed channel 362 is simultaneously the feed opening to the subsequent main space 352.

Formed at the rear of the filter medium 354 from the perspective of the main space 352 is a respective filtrate space or rear space 356 from which one or more filtrate channels 364 lead to one of the four collecting channels. The discharging of the filtrate is effected via the end element 342 (head element) shown on the left-hand side which also effects the feeding of the substance mixture.

According to the invention two discharging openings 376 by means of which the liquefaction medium and the liquefied valuable material are discharged and supplied to the downstream processor unit 200 are provided at each main space 354. Each discharge opening 376 is connected to a discharge channel 378 which is in turn connected to the return conduit 208 shown in FIGS. 1 and 2.

It is thus possible for regeneration of the membrane filter press 340 after filling thereof to produce a short circuit flow for the liquefaction medium which leads via the feed channel 362 and in every filter element 344 via the feed opening 374 and the respective main chamber 352 to the discharge opening 376 into common discharge channels 378.

### EXEMPLARY EMBODIMENT

In the process for producing methionine (MET) described at the outset in the inventive configuration in reactor 201 methionine hydantoin saponification is carried out according to formula 1.

Subsequently in reactor 202 methionine is liberated from its alkali metal salt by carbonation with carbon dioxide according to the second formula and filtered off as solid from the substance mixture containing alkali metal carbonate and alkali metal hydrogencarbonate (e.g. potassium carbonate and potassium hydrogencarbonate) in the first filter apparatus 302.

Prior to the first carbonation the substance mixture in the feed conduit 204 still comprises 119 to 151 g/kg of active potassium cations (a.K+), for example in the form of KHCO3, K2CO3, and 7.1 to 9.0 g/kg of formate as the potassium salt of formic acid. The formate is an inhibitor or contaminant for the methionine process and it is advantageous to remove it as completely as possible.

After the first separation apparatus 302 the filtrate has a concentration of 61.5 to 78.5 g/kg of a.K+ and 8.8 to 11.2 g/kg of formate and is subsequently subjected to thermal concentration in an evaporator (not shown). The substance mixture accordingly has a concentration of 114.5 to 145.5 g/kg of a.K+ and 15.8 to 20.2 g/kg of formate upon entry into reactor the 330.

Finally, the valuable material methionine, potassium carbonate, potassium hydrogencarbonate and a proportion of the byproduct such as for example methionyl-methionine (met-met) is filtered out in the second separation apparatus to obtain a highly concentrated product.

The plant for methionine production is configured according to FIG. 2 and may comprise further subunits comprising a precipitation reactor and a filter apparatus.

The valuable material methionine comprehensively depleted in this way, potassium carbonate, potassium hydrogencarbonate and the byproduct met-met may be recycled in liquefied form via the conduit 208 without any adverse effects on the process unit 200.

By eliminating the need for opening, cleaning, and resealing of the second, terminal separation apparatus 304 which is configured as a membrane filter press, a marked performance enhancement of the overall process for methionine production was achieved.

A low apparatus complexity is especially achieved by the omission of a separate process unit for removing and transporting away the filter cake which would need to be performed in encapsulated fashion to avoid condensate formation and odour entrainment into the adjacent plant. Furthermore, the mechanical wear on the components of the filter press is reduced since a very frequent plate motion per filtration cycle is omitted. Plate transport at the filter press can be largely eliminated. Finally, the filter fabric is also protected, the service life is extended and clogging of the filter medium is largely minimized. FIG. 4 shows in two partial views A), B) an alternative embodiment of a membrane filter frame 370 (partial view A) and an associated filter frame 372 (partial view B), wherein the desired vertical orientation is shown. In contrast to the previous embodiment of FIG. 3 the feed channel 362/the two feed channels 362 are arranged at the lower frame section 346 and the discharge channel 378 is positioned centrally at the upper frame section 348. For introduction of the suspension from the feed channel 362 the filter frames 372 each have shaft- or slot-like feed openings 374 as well as a corresponding discharge opening 376. Since these feed openings 374 and the discharge openings 376 pass the respective substance mixture over the edge of the respective filter medium 354 it is provided in one embodiment that is not further specified that the feed and discharge openings 374, 376 are in the form of a completely or partially closed shaft or channel element which extends from the feeder channel 362 to beyond the edge of the inserted filter medium 354 and for example additionally secure the filter medium 354 by clamping. The operation of the membrane filter press 340 according to the embodiment in FIG. 4 or 5 is described in detail in connection with FIG. 6.

FIG. 5 shows a membrane filter press 340 analogous to FIG. 4. As in FIG. 4 the main space 352 is formed on one side by the membrane filter frame 370 (partial representation A) and on the accompanying other side by the rigid filter frame 372 (partial representation B) which are shown in an open position. The main space 352 has an octagonal basic shape. Two segments of the feed channels 362 are arranged at the lower frame section 346 and a discharge channel 378 is provided at the upper frame section 348. Furthermore, a plurality of filtrate channels 364 that are connected to one of the collecting channels 366 for the filtrate lead out of the main space 352. A respective feed opening 374 leads from each of the feed channels 362 into the main space 352. In analogous fashion a discharge opening 376 leads from the main space 352 into the discharge channel 378. These feed and discharge openings 374, 376 may be configured as described in connection with FIG. 4.

It is common to the embodiments of FIGS. 4 and 5 that both the suspension to be filtered (feed) and the liquefaction medium are introduced via the lower feed channels 362.

The advantage of this embodiment according to FIGS. 4 and 5 is inter alia that when introducing a liquefaction medium into the main space 352 a defined flow is formed and fragments of the filter cake fall towards the inflowing, fresh liquefaction medium. Smaller fragments are sifted by the ascending flow and upon reaching a sufficiently small size are entrained.

FIG. 6 shows an end element 342 of a membrane filter press 340 and all feed and discharge options for the different media. FIG. 6 shall hereinbelow serve to describe in detail the individual process steps for operating the membrane filter press 340.

Starting with an empty membrane filter press 340:
(1) Introduction of the suspension S from below via the two feed channels 362, wherein simultaneously the trapped gas A, generally air, is released via the upper collecting channels 366 and/or separate venting conduits and wherein the discharge channel 378 is closed (not shown) until the main chambers 352 are completely filled. The discharge channel 378 may temporarily be utilized for discharging the gas A.
(2) Further introduction of suspension S into the main space via the feed channels 362, first compression of the filter cake by inflowing suspension S, wherein the filtrate FT is discharged via the collecting channels 366.
(3) Complete filling of the main space 352 with a filter cake and end of introduction of suspension S, introduction of gas A into the main space 352 via the upper collecting channels 366 for more far-reaching dewatering of the filter cake and discharging of the filtrate FT via the lower collecting channels 366.
(4) Introduction of a fluid, in particular water, into the interior of the membrane filter frame 370 via the port 380 (shown as double arrow B), which may also be arranged at the top, and compression and final dewatering of the filter cake, wherein the compression of the filter cake and resetting of the membrane brings about a flow path or flow slot in the main space 352 between the (restored) membrane and at least one side of the filter cake.
(5) Introduction of the unladen liquefaction medium VM from below via the two feed channels 362 and discharging of the laden liquefaction medium VM+ via the upper discharge channel 378 until the filter cake is completely liquefied/sufficiently small fragments are dischargeable.
(6) Introduction of gas A, in particular compressed air (shown as a double arrow), via the upper collecting conduits 366 into the main space 352, optional blocking of the upper discharge channel 378. Discharging of the waiting laden liquefaction medium VM+ by introduction of gas A and pressure elevation in the main chamber 352 in counter current via the feed channels 362 until the laden liquefaction medium VM+ is completely displaced. In this case the displaced liquefaction medium VM+ may be collected in a container, for example a container of the receiving and conditioning unit 400. Purification or filtration of the discharged gas A when this is laden for example with volatile proportions or hazardous substances of the liquefaction medium VM as required.
(7) Introduction of suspension S according to step (1), in particular without preceding opening of the membrane filter press.

The opening and blocking of the active and non-active channels was not performed separately and is apparent to a person skilled in the art analogously from the recited desired flow management/direction.

In the present invention the terms “suspension” and “substance mixture to be separated” are used synonymously.

In the present invention the term “liquefaction medium” in particular relates to a substance mixture, a suspension, or a filtrate or to an acid or alkali which is used for the purpose of liquefying the solid valuable material and is optionally conditioned therefor. Furthermore, the term “suspension” is to be understood as meaning a heterogeneous mixture of a (continuous) fluid and a (disperse) solid distributed therein.

### LIST OF REFERENCE SYMBOLS

100 Plant
200 process unit
201 Reactor
202 Reactor
204 Feed conduit
206 Connecting conduit (to 300, 302)
208 Return conduit (from 304 to 200)
210 Data line
212 Return conduit (from 232, 208 into 200)
214 (Regeneration) conduit
216 Outflow
220 Valve unit
222 Valve unit
224 Valve unit (from 400)
226 Valve unit
230 Container
232 Container
300 Separation unit
302 Separating apparatus, first
304 Separating apparatus, second
306 Application unit
308 Conveyor unit, belt filter
310 Vacuum apparatus
312 Discharge (from 304)
314 Conduit (from 302, 310 to 304)
316 Conduit (from 400 via 222 to 304)
318 Discharge (from P from 302)
320 Container
322 Container
324 Conveying means, pump
326 Bypass conduit
330 Reactor
340 Membrane filter press
342 Edge/end element
344 Filter element
346 Frame section, lower
348 Frame section, upper
350 Frame section, lateral
352 Main space
354 Filter medium
356 Filtrate/rear space
358 Interior
360 Membrane
362 Feed channel (from S)
364 Filtrate channel (for FT)
366 Collecting channel (for FT, 364)
370 Membrane filter frames
372 Filter frames
374 Feed opening (to 352 in filter frame)
376 Discharge opening (for S)
378 Discharge channel (for S after 376)
380 Ports (for introduction of B)
400 Receiving and conditioning unit
410 (Receiving) tank
412 Conduit
414 Connecting conduit
420 Heat exchanger
422 Heat exchanger
500 Control unit
A Fluid1: Gas, air
B Fluid2: Water
E Reactant
FT Filtrate
G Gravitational direction
S Suspension
VM Liquefaction medium
VM+ Liquefaction medium, laden with valuable material

1. Plant (100) for producing at least one product comprising a first process unit (200) and at least one connected separating unit (300), wherein the process unit (200) comprises the following:
at least one reactor (201, 202) for treating a substance mixture,
at least one feed conduit (204) for introducing at least one reactant into the at least one reactor (201, 202),
a connecting conduit (206) for discharging a suspension comprising at least one precipitated, solid product to the separating unit (300), wherein the separating unit (300) is configured for separating the at least one solid product and discharging of at least one harmful byproduct or inhibitor and the separation unit (300) comprises two (302, 304), three or four separation apparatuses with at least two separation apparatuses (302, 304) being serially connected via
a conduit (214, 314) to a receiving and conditioning unit (400) configured for conditioning a liquefaction medium to liquefy valuable material collected as a filter cake in the membrane filter press (340), and via
a return conduit (208) leading to the process unit (200) to recycle the at least one liquefied valuable material.

2. Plant according to claim 1, characterized in that the membrane filter press (302) comprising two end elements (342) and a plurality of filter elements (344), wherein a filter space which is dividable into a main space (352) and a rear back or filtrate space (356) by a filter medium (354) is in each case formed between two filter elements (342, 344, 370, 372) and associated frame sections (346, 348, 350), wherein at least one filter element (344) is formed as a membrane filter frame (370) by two filter elements (344) forming a main space (352) and comprises a fluidizable interior (358) which is delimited by at least one flexible membrane (360) in the direction of the filter space and wherein the main space (352) has at least one feed opening (374), in particular a feed opening (374) leading to at least one feed channel (362), for a substance mixture to be separated and the back or filtrate space (356) has at least one filtrate channel (364) for the filtrate (FT), in particular at least one filtrate channel (364) which leads to a collecting channel (366), characterized in that the main space (352) comprises not only the feed opening (374) for a substance mixture but also at least one discharge opening (376), in particular a discharge opening (376) arranged vertically above the at least one feed channel (362).

3. Plant according to claim 1, characterized in that the membrane filter press (340) comprises a plurality of filter elements (344), wherein a main space (352) is in each case formed between two adjacent filter elements (304), wherein at least two main spaces (352) are connected to at least one discharge channel (378) of the membrane filter press (340) via the in each case one discharge opening (376) and wherein the discharge channel (378) is connected to the process unit (200) via a return conduit (208).

4. Plant according to claim 2, characterized in that the feed channels (362) and the connected main spaces (352) of the membrane filter press (340) are connected
I. to a filtrate-conducting conduit (314) of an upstream separation apparatus (302),
II. via a flow path (214) (214, 314) to the outflow (216) of the process unit (200) and/or
III. to a receiving and conditioning unit (400) for providing a liquefaction medium.

5. Plant according to claim 1, characterized in that the separating unit (300) comprises two, three or four subunits comprising a precipitation reactor (330) and a subsequent filter apparatus (304).

6. Plant according to claim 2, characterized in that the at least one feed channel (362) of the membrane filter press (340) is arranged at a vertically lower frame section (346).

7. Plant according to claim 2, characterized in that the at least one discharge opening (376) of the membrane filter press (340), arranged vertically above the feed channel (362) is arranged at the vertically upper frame section (348).

8. Apparatus according to claim 3, characterized in that the number of the lower feed channels (362) of the membrane filter press (340) is at least one greater than the number of the discharge channels (378) arranged above.

9. Plant according to claim 3, characterized in that the ratio of the sum of the flow cross sections of the feed channels (362) to the sum of the flow cross sections of the discharge channels (378) is in the range from 1:3 to 2:1, ideally in the range from 1:2 to 1:1.

10. Process for producing at least one solid product in a plant (100), comprising the following steps:
a. provision of a process unit (200) and a separating unit (300),
b. introduction of at least one reactant into the process unit (200) and production of at least one solid product and byproduct in a substance mixture,
c. separation of the at least one solid product and discharging of at least one harmful byproduct or inhibitor using two (302, 304), three or four separation apparatuses in the separating unit (300), wherein at least two separation apparatuses (302, 304) are serially connected and the following steps are performed:
d. collection of a first sub-amount of the separable solid product as filter cake in the first separation apparatus (302),
e. dehumidification of the first sub-amount of the solid product in the first separation apparatus (302),
f. sending the filtrate (FT) separated in the first separation apparatus (302) and comprising valuable material selected from reactants, byproducts, auxiliaries and/or end products to the last separation apparatus (304) which is configured as a membrane filter press (340),
g. collection of a separable valuable material as filter cake in the last separation apparatus (304), and
h. dehumidification of the valuable material in the last separation apparatus (304),
i. liquefaction of the valuable material collected in the membrane filter press (340) and solidified as a filter cake inside the membrane filter press (340) with a liquefaction medium, and
j. recycling of the liquefaction medium with the valuable material dissolved therein into the process unit (200).

11. (canceled)

12. Process according to claim 10, characterized in that the dehumidification in the first step is carried out to a residual moisture content in the separated solid product of down to 30% and the dehumidification in the last step is carried out to a residual moisture content in the separated valuable material of not more than 30%.

13. Process according to claim 10, characterized in that the liquefaction medium for liquefaction of the valuable material collected inside the membrane filter press (340) is a substance mixture, a suspension and/or a filtrate from an upstream part of the plant and/or a preceding process step, in particular a conditioned substance mixture, a suspension and/or a filtrate.

14. Process according to claim 10, characterized in that the liquefaction medium for liquefaction of the valuable material collected inside the membrane filter press (340) is an acid or an alkali which is not a substance mixture, not a suspension and/or not a filtrate from an upstream part of the plant and/or a preceding process step.

15. Process according to claim 10, characterized in that during the liquefaction the (separated) valuable material collected inside the membrane filter (340) is subjected to a flow of the liquefaction medium from below in the gravitational direction and is discharged from the respective main space (352) and the respective filter element (344) at the top and/or above the feed channel (362) in the gravitational direction.

16. Process according to claim 10, characterized in that the plant (100) for producing the at least one product or valuable material comprises a first process unit (200) and at least one connected separating unit (300), wherein the process unit (200) comprises the following:
at least one reactor (201, 202) for treating a substance mixture,
at least one feed conduit (204) for introducing at least one reactant into the at least one reactor (201, 202),
a connecting conduit (206) for discharging a suspension comprising at least one precipitated, solid product to the separating unit (300), wherein the separating unit (300) is configured for separating the at least one solid product and discharging of at least one harmful byproduct or inhibitor and the separating unit (300) comprises two (302, 304), three or four separation apparatuses with at least two separation apparatuses (302, 304) being serially connected via a conduit (314) and the last separation apparatus being a membrane filter press (340),
characterized in that
the membrane filter press (340) is connected
via a conduit (214, 314) to a receiving and conditioning unit (400) configured for conditioning a liquefaction medium to liquefy valuable material collected as a filter cake in the membrane filter press (340), and
via a return conduit (208) leading to the process unit (200) to recycle the at least one liquefied valuable material.

17. Process according to claim 10, characterized in that the process is used for producing methionine.

18. Process according to claim 17, characterized in that at least two precipitation steps are carried out, wherein before at least two of the separation steps a precipitation step is carried out, and wherein at least one of the precipitation steps, in particular both precipitation steps, is a carbonation step, in particular a carbonation using CO₂.

19. (canceled)

20. Process according to claim 10, characterized in that after the dehumidification the membrane filter press (340) is emptied and/or regenerated by means of a liquefaction of the filter cake, in particular by means of an emptying and/or regeneration with a closed membrane filter press (340), and wherein after the liquefaction of the filter cake, the liquefaction medium laden with valuable material is discharged via at least one internal discharge channel (378) of the membrane filter press (340).
